(19) United States
(12) Patent Application Publication
(10) Pub. No.: US 2017/0014313 A1
(43) Pub. Date: Jan. 19, 2017

(54) HYGIENE PRODUCT TABLET AND METHODS OF FORMING SAME

(71) Applicant: Nohbo, LLC, Reston, VA (US)

(72) Inventors: Benjamin Gabriel STERN, Reston, VA (US); Robert Hutton Ray, Union, IL (US)

(21) Appl. No.: 15/211,788

(22) Filed: Jul. 15, 2016

Related U.S. Application Data
(60) Provisional application No. 62/192,679, filed on Jul. 15, 2015.

(51) Int. Cl. A61K 8/02; A61K 8/46; A61K 8/34; A61Q 19/10; A61K 8/73; A61Q 9/02; A61Q 5/02; A61K 8/60; A61K 8/97

(52) U.S. Cl. CPC A61K 8/0216; A61K 8/60; A61K 8/466; A61K 8/345; A61K 8/97; A61K 8/732; A61Q 9/02; A61Q 5/02; A61Q 19/10; A61K 2800/592; A61K 2800/49

(57) ABSTRACT

A hygiene product tablet an method of forming the same, the tablet including: an active agent; a binder; a non-aqueous wetting agent; and a binder enhancer, which are compressed in a mold at a pressure ranging from about 100 psi to about 180 psi. The tablet may include a core and a shell surrounding the core, the shell having a higher amount of cross-linking than the core.

### RELATED APPLICATIONS

This application claims the benefit of priority to U.S. Provisional Application No. 62/192,679, entitled “HYGIENE PRODUCT TABLET AND METHODS OF FORMING SAME”, filed Jul. 15, 2015, the entire contents of which is incorporated herein by reference.

### FIELD

Aspects of the present disclosure provide a hygiene product tablet configured for single-use applications, and a method of forming such a tablet.

### BACKGROUND

Hygiene products, such as shampoos, bodywashes, shaving cream, and conditioners are usually sold in a liquid or gel format. Such hygiene products generally contain active agents, such as surfactants and/or conditioners, in addition to significant amounts of water and/or viscosity control agents. Such hygiene products are most commonly provided in bottles containing enough product for many applications. While such products are suitable for many consumer applications, there is a need for smaller amounts of hygiene product, particularly in the travel and hospitality industries.

Hygiene products have been provided in small bottles for use in the hospitality and/or travel industries. However, the small bottles of shampoo/conditioner/shaving cream generally found in the hospitality industry have a high packaging to product ratio, which contributes to higher costs and excessive amounts of waste.

In addition, single-dose packages of shampoo/conditioner packaged in plastic sachets, bags, or blister packs have also been developed. However, such packaging is generally not recycled or biodegradable. A hygiene product in a solid form could be packaged in a more environmentally friendly material, and could also reduce packaging waste. However, conventional solid hygiene products suffer from slow dissolution speed and a lack of public acceptance.

For example, U.S. Pat. No. 4,330,438 describes a powder shampoo concentrate comprising a mixture of an anionic surfactant and a nonionic derivative of a polygalactomannan gum together with conventional shampoo ingredients. U.S. Pat. No. 6,451,297 describes a hair care product in the form of a powder having a granulometry of 30 to 500 microns, applicable directly to the wet hair and/or the body and comprising less than 40% of at least one surfactant, and from 1 to 12% of at least one perfume, the percentage being made up to 100% by one or more products selected from the group consisting of sugars, starches, celluloses, polyols, proteins, amino acids, perfumes, colorings, antioxidants, plant substances, seaweed, vitamins, essential oils and mineral fillers. Such powder shampoos are prepared by blending powder raw materials in powders. This approach forms powders which do not dissolve readily enough and tend to give some grains upon dissolution. Further, only solid raw materials can be incorporated.

### SUMMARY

Exemplary embodiments of the present disclosure provide a single-use hygiene product tablet. In various embodiments, the hygiene product tablet may include, based on the total weigh of the tablet: a binder in an amount ranging from about 12 wt % to about 20 wt %; an active agent in an amount ranging from about 55 wt % to about 70 wt %; and a non-aqueous wetting agent in an amount ranging from about 7 wt % to about 18 wt %.

Exemplary embodiments of the present disclosure provide a method of forming a hygiene product tablet, the method including: forming a first mixture by mixing a binder and an active agent; forming a second mixture by mixing a non-aqueous wetting agent into the first mixture; compressing the second mixture in a mold at from about 100 psi to about 180 psi, to form a tablet; and coating the tablet with water or an aqueous mixture including a hardening agent.

Exemplary embodiments of the present disclosure provide a single-use hygiene product tablet including: a binder; a sulfate-free surfactant; a non-aqueous wetting agent; and a cross-linkable, hygroscopic polymer. The tablet may have a core and a shell surrounding the core, the shell having a higher degree of cross-linking than the core, and the tablet may be formed by compression in a mold, at a pressure ranging from about 100 psi to about 180 psi.

### BRIEF DESCRIPTION OF THE DRAWINGS

The accompanying drawings, which are incorporated herein and constitute part of this specification, illustrate exemplary embodiments of the invention, and together with the general description given above and the detailed description given below, serve to explain the features of the invention.

FIGS. 1A-1C are sectional views of hygiene tablet pellets, according to various embodiments of the present disclosure

FIG. 2 is a block diagram illustrating a method of forming hygiene product tablets, according to various embodiments of the present disclosure.

FIG. 3 is a block diagram illustrating a method of forming hygiene product tablets, according to various embodiments of the present disclosure.

FIG. 4 is a block diagram illustrating a method of forming hygiene product tablets, according to various embodiments of the present disclosure.

FIGS. 5A-5D are photographs showing steps of the method of FIG. 4.

FIGS. 6A-6C are photographs showing hygiene product tablets produced by the method of FIG. 4.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

The word “exemplary” is used herein to mean “serving as an example, instance, or illustration.” Any implementation described herein as “exemplary” is not necessarily to be construed as preferred or advantageous over other implementations.

Various embodiments relate to hygiene product tablets provided in a single dose format. The hygiene product tablets may operate as cleansing and/or conditioning agents for hair and/or skin.

Various embodiments include hygiene products in the form of single-dose tablets. As referred to herein, a “tablet” may refer to pellet, capsule, sphere, or the like. The tablets may have any suitable shape and/or size. Te tablets may contain a sufficient amount of hygiene product for a single use. For example, a tablet may contain an amount of shampoo sufficient to clean one head of hair, may contain an amount of body wash sufficient to wash one body, or may contain an amount of shaving cream sufficient to shave one or more body parts.

The tablets may be solid, semi-solid, or in the form of an encapsulated liquid/gel. The tablets may be compressed to assume a suitable shape and/or density. The tablets may be configured to rapidly dissolve in the presence of a solvent such as water. Weight percentages given herein for tablet components are based on the total weight of the tablet.

According to various embodiments, the hygiene product tablets may include at least one active agent, at least one binding agent, at least one wetting agent, and may optionally include at least one secondary ingredient. The active agent may be dispersed in the binding agent. The binding agent may include a binder and a binder enhancer. The binder, binder enhancer, and/or wetting agent may be configured to increase the integrity of a tablet and/or may operate to disperse the active agent when the tablet is dissolved in water.

Active Agents

In various embodiments, the active agent may include a shampoo, a bodywash, a conditioner, or a combination thereof. In other embodiments, the active agent may include a shampoo concentrate or a bodywash concentrate. In some embodiments, the active agent may include a powdered surfactant or powered surfactant mixture. In other embodiments, the active agent may include shaving cream or gel.

In various embodiments, the active agent may include an anionic, nonionic, or amphoteric surfactant, or combinations thereof. In various embodiments, a tablet may contain a surfactant in an amount ranging from about 8 wt % to about 25 wt %, such as from about 10 wt % to about 23 wt %, or from about 12 wt % to about 20 wt %.

Examples of suitable anionic surfactants include alkali metal sulforicinates, sulfonated glyceryl esters of fatty acids, such as sulfonated monoglycerides of coconut oil acids, salts of sulfonated monovalent alcohol esters such as sodium oleylisethianate, metal soaps of fatty acids, amides of amino sulfonic acids such as the sodium salt of oleyl methyl tauride, sulfonated products of fatty acids nitriles, such as palmitonitrile sulfonate, sulfonated aromatic hydrocarbons such as sodium alpha-naphthalene monosulfonate, condensation products of naphthalene sulfonic acids with formaldehyde, sodium octahydroanthracene sulfonate, alkali metal alkyl sulfates such as sodium lauryl sulfate, ammonium lauryl sulfate or triethanolamine lauryl sulfate, ether sulfates having alkyl groups of 8 or more carbon atoms, such as sodium lauryl ether sulfate, ammonium lauryl ether sulfate, sodium alkyl aryl ether sulfates, and ammonium alkyl aryl ether sulfates, alkylarylsulfonates having 1 or more alkyl groups of 8 or more carbon atoms, alkylbenzenesulfonic acid alkali metal salts exemplified by hexylbenzenesulfonic acid sodium salt, octylbenzenesulfonic acid sodium salt, calcium salts, decylbenzenesulfonic acid sodium salt, dodecylbenzenesulfonic acid sodium salt, cetylbenzenesulfonic acid sodium salt, and myristylbenzenesulfonic acid sodium salt, sulphuric esters of polyoxyethylene alkyl ether including CH₃(CH₂)₆CH₂O(C₂H₄O)₂SO₃H, CH₃(CH₂)₇CH₂O(C₂H₄O)_{3.5}SO₃H, CH₃(CH₂)₈CH₂O(C₂H₄O)₈SO₃H, CH₃(CH₂)₁₉CH₂O(C₂H₄O)₄SO₃H, and CH₃(CH₂)₁₀CH₂O(C₂H₄O)₆SO₃H, sodium salts, potassium salts, and amine salts of alkylnapthylsulfonic acid.

Examples of cationic surfactants include various fatty acid amines and amides and their derivatives, and the salts of the fatty acid amines and amides. Examples of aliphatic fatty acid amines include dodecylamine acetate, octadecylamine acetate, and acetates of the amines of tallow fatty acids, homologues of aromatic amines having fatty acids such as dodecylanalin, fatty amides derived from aliphatic diamines such as undecylimidazoline, fatty amides derived from aliphatic diamines, such as undecylimidazoline, fatty amides derived from disubstituted amines such as oleylaminodiethylamine, derivatives of ethylene diamine, quaternary ammonium compounds and their salts which are exemplified by tallow trimethyl ammonium chloride, dioctadecyldimethyl ammonium chloride, didodecyldimethyl ammonium chloride, dihexadecyl ammonium chloride, alkyltrimethylammonium hydroxides such as octyltrimethylammonium hydroxide, dodecyltrimethylammonium hydroxide, or hexadecyltrimethylammonium hydroxide, dialkyldimethylammonium hydroxides such as octyldimethylammonium hydroxide, decyldimethylammonium hydroxide, didodecyldimethylammonium hydroxide, dioctadecyldimethylammonium hydroxide, tallow trimethylammonium hydroxide, trimethylammonium hydroxide, methylpolyoxyethylene cocoammonium chloride, and dipalmityl hydroxyethylammonium methosulfate, amide derivatives of amino alcohols such as beta-hydroxylethylstearylamide, and amine salts of long chain fatty acids.

Examples of suitable cationic surfactants include also quaternary ammonium halides such as octyl trimethyl ammonium chloride, dodecyl trimethyl ammonium chloride, hexadecyl trimethyl ammonium chloride, octyl dimethyl benzyl ammonium chloride, decyl dimethyl benzyl ammonium chloride and coco trimethyl ammonium chloride as well as other salts of these materials, fatty amines and basic pyridinium compounds, quaternary ammonium bases of benzimidazolines, polypropanolpolyethanol amines, polyethoxylated quaternary ammonium salts and ethylene oxide condensation products of the primary fatty amines, available from Armak Company, Chicago, Ill. under the tradenames Ethoquad, Ethomeen, or Arquad. Suitable cationic surfactants can also be an esterquat type compound.

Examples of nonionic surfactants include decyl glucosides, polyoxyethylene alkyl ethers, polyoxyethylene alkylphenol ethers, polyoxyethylene lauryl ethers, polyoxyethylene sorbitan monoleates, polyoxyethylene alkyl esters, polyoxyethylene sorbitan alkyl esters. Suitable nonionic surfactants include condensates of ethylene oxide with a long chain (fatty) alcohol or (fatty) acid, condensates of ethylene oxide with an amine or an amide, condensation products of ethylene and propylene oxides, fatty acid alkylol amide and fatty amine oxides. Examples of non-ionic surfactants include polyoxyalkylene alkyl ethers such as polyethylene glycol long chain (12-14C) alkyl ether, polyoxyalkylene sorbitan ethers, polyoxyalkylene alkoxylate esters, polyoxyalkylene alkylphenol ethers, ethylene glycol propylene glycol copolymers, polyvinyl alcohol and alkylpolysaccharides.

The amphoteric surfactants can be, in particular (non-limiting list), aliphatic secondary or tertiary amine derivatives in which the aliphatic radical is a linear or branched chain containing 8 to 22 carbon atoms and containing—at least one water-soluble anionic group (for example carboxylate, sulphonate, sulphate, phosphate or phosphonate); mention may also be made of (C₈-C₂₀)alkyl-betaines, sulphobetaines, (C₈-C₂₀)alkylamido(C₁-C₆)alkyl-betaines or (C₈-C₂₀)alkylamido(C₁-C₆)alkylsulphobetaines.

In some embodiments, environmentally friendly surfactants may be used, such as sulfate-free surfactants. For example, the surfactant may be sodium cocoyl isethionate, sodium lauryl sulfoacetate, alpha olefin sulfonate, or the like, or combinations thereof. When tablets that included more than one sulfate-free surfactant were dissolved in water, flash foaming effects were unexpectedly produced.

Non-limiting examples of useful conditioners include petrolatum, fatty acids, esters of fatty acids, fatty alcohols, ethoxylated alcohols, polyol polyesters, glycerin, glycerin mono-esters, glycerin polyesters, epidermal and sebaceous hydrocarbons, lanolin, straight and branched hydrocarbons, silicone oil, silicone gum, vegetable oil, vegetable oil adduct, hydrogenated vegetable oils, nonionic polymers, natural waxes, synthetic waxes, polyolefinic glycols, polyolefinic monoester, polyolefinic polyesters, cholesterols, cholesterol esters, triglycerides and mixtures thereof.

Binders

In some embodiments, a binder included in the binding agent may include saccharides and their derivatives, proteins, and/or synthetic polymers, combinations thereof, or the like. The saccharides may include disaccharides such as sucrose and lactose, polysaccharides and their derivates, such as starches and celluloses, and sugar alcohols, such as xylitol, sorbitol, and malititol. Other useful binders may include partially pre-gelatinized corn starch, or the like, tapioca maltodextrin, beta-glucan, dosium tartarate, benotonite clay, maltodextrin, sodium bicarbonate, alovera polysaccharides, combinations thereof, or the like.

In some embodiments, a tablet may include a binder in an amount ranging from about 50 wt % to about 75 wt %, such as from about 55 wt % to about 70 wt %. In some embodiments, a tablet may contain a binder and surfactant in an amount ranging from about 65 wt % to about 90 wt %, such as from about 70 wt % to about 88 wt %.

Binder Enhancers

In some embodiments, the binder enhancer may include swellable, cross-linkable, hygroscopic polymers, such as guar gum (e.g., cyamopsis tetragonoloba gum), cationic guar gum, xanthan gum, or the like, or combinations thereof. In some embodiments, tablets may include a hygroscopic polymer in an amount ranging from about 0 wt % to about 0.4 wt %, such as from about 0.05 wt % to about 0.3 wt %, or from about 0.1 wt % to about 0.2 wt %.

In some embodiments, the binder enhancer may include starch, pregelatinized starch, a modified starch, such as hydroxypropyl starch phosphate and sodium starch glycolate, combinations thereof, or the like. For example, a tablet may include modified starch in an amount ranging from about 0.1 wt % to about 3 wt %, such as from about 0.2 wt % to about 2 wt %, or from about 0.3 wt % to about 1 wt %.

In some embodiments, the binder enhancer may include honey, polyvinyl pyrolidone (PVP), ExpertGel EG312 (poloxamer 338/PPG-12/SMDI copolymer) available from DKSH Inc., ExpertGel EG412 (poloxamer 407, PPG-12/SMDI copolymer) available from DKSH Inc, or any combination thereof.

The combination of the binder and the binder enhancer may impart improved particle adhesion and compression characteristics. The combination of the binder and binder enhancer may also impart excellent dispersability.

In some embodiments, the binding agent may include a gellant and a gel activator. The gellant and gel activators may work together to at least partially gelate the hygiene product. In some embodiments, the gellant may include an ion-activated polysaccharide. For example, ion-activated polysaccharide may include iota carageenan, kappa carageenan, and lambda carageenan, sodium alginate, or a combination thereof.

The gel activator may include various ions or salts thereof. For example, the gel activator may include calcium ions, potassium ions, or hydroxide ions, any combination thereof, or any salts or compounds capable of generation such ions. The gel activator ions may be blended at various ratios, in order to provide a suitable pH for activating the gellant. In some embodiments, the gel activator may be water, such as when a hydration-activated gellant/binder is included in a tablet.

In some embodiments, the gellant and gel activators may be dispersed throughout the tablet. In some embodiments, the gellant and gel activator may form a capsule/membrane around the active agent.

Wetting Agents

In various embodiments, the wetting agent may be a non-aqueous liquid. The wetting agent may also be configured to improve tablet binding and compaction by moistening tablet components. For example, the wetting agent may be configured to activate polar cross-linking between polymers dispersed in the binder, such as the above binder enhancers. The wetting agent may also be configured to act as a disintegrant when a tablet is dissolved in water.

In some embodiments, the wetting agent may include glycerin, propylene glycol, dipropylene glycol, N-Acetyl Diglycoamine, safflower oil, combinations thereof, or the like. In some embodiments, an amount of wetting agent included in a tablet may range from about 2 wt % to about 20 wt %, such as from about 7 wt % to about 18 wt %, or about 8 wt % to about 16 wt %.

Secondary Ingredients

In various embodiments, the hygiene product tablets may include secondary ingredients. For example, the secondary ingredients may include dyes/colorants, fragrances, texture modifiers, foam enhancers, disintegrants, compaction enhancers, anti-caking agents, hardening agents, anti-microbial agents, combinations thereof, or the like.

Secondary ingredients useful for improving compaction, dissolvability, and foaming may include coconut milk powder, arrow root powder, colloidal oatmeal powder, combinations thereof, or the like. In various embodiments, a tablet may include a secondary ingredient, such as coconut milk powder, arrow root powder, colloidal oatmeal powder, or any combination thereof, in an amount ranging from about 0 wt % to about 22 wt %, such as from about 5 wt % to about 20 wt %, or from about 8 wt % to about 15 wt %. Colloidal oatmeal powder may also operate as a texture modifier to provide a smoother texture, and may also operate as a binding enhancer.

Any suitable colorant may be used. A tablet may include a colorant in an amount ranging from about 0 wt % to about 0.3 wt %, such as from about 0.1 wt % to about 0.2 wt %.

In various embodiments, a suitable fragrance may be used. Useful fragrances may be in liquid form, such as in the form of fragrance oils. A tablet may include a fragrance oil in an amount ranging from about 0 wt % to about 0.3 wt %, such as from about 0.1 wt % to about 0.2 wt %.

In various embodiments, texture modifiers may include micro or macro abrasive agents, such as when a tablet includes a body scrub. Suitable abrasive agents include, for example, nut powders, silica powders, polymer beads such as wax beads, combinations thereof, or the like. In some embodiments, a tablet may include an abrasive agent in an amount ranging from about 0 wt % to about 3 wt %, such as from about 0.1 wt % to about 2 wt %, or from about 0.2 wt % to about 1 wt %.

In some embodiments, hardening agents may include witch hazel, hydrated urea, combinations thereof, or the like. The hardening agent may be applied to the surface of a tablet. For example, the hardening agent may be dispersed in an aqueous solution and coated onto a tablet. A tablet may include a hardening agent in an amount ranging from about 0 wt % to about 0.2 wt %, such as from about 0.01 wt % to about 0.1 wt %, or from about 0.02 wt % to about 0.05 wt.

In various embodiments, anti-microbial agents may be natural materials having anti-microbial effects. For example, anti-microbial agents may include thyme oil, tea tree oil, oregano oil, lavender oil, citrus essential oil, grapefruit seed extract, olive leaf extract, honey, or the like. A tablet may include an anti-microbial agent in an amount ranging from about 0 wt % to about 3 wt %, such as from about 0.1 wt % to about 2 wt %, or from about 0.2 wt % to about 1 wt %.

In various embodiments, secondary ingredients may be mixed into tablet compositions, may be applied to tablets via a water bath in which the tablets are submerged as discussed below, or may be sprayed onto tablets.

In some embodiments, a tablet containing shampoo, conditioner, or a combination thereof, may range in volume from about 4 g to about 12 g, such as from about 5 g to about 8 g. A tablet containing body wash may range in volume from about 6 g to about 15 g, such as from about 8 g to about 10 g. Tablets including a concentrated active agent may be smaller than tablets containing undiluted or semi-diluted active agents.

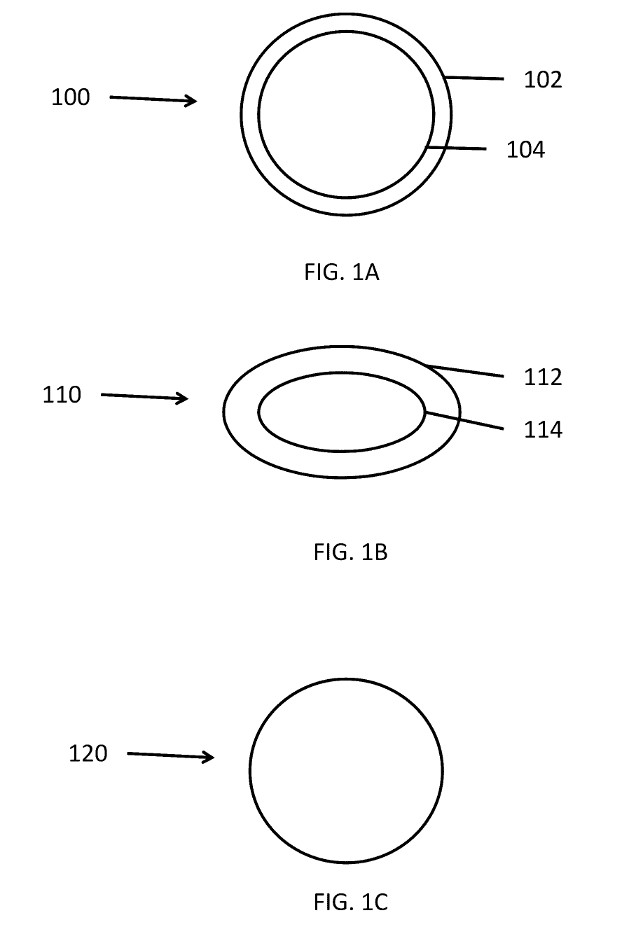

FIGS. 1A, 1B, and 1C are respectively cross-sectional views of hygiene product tablets 100, 110, and 120, according to various embodiments. Referring to FIG. 1A, the tablet 100 is generally spherical and includes a core 104 surrounded by an envelope 102. Herein, the envelope may also be referred to as a shell The envelope 102 and the core 104 may be structurally or chemically distinct layers as shown in FIG. 1A.

In particular, the envelope 102 and the core 104 may be formed of different materials. For example, the envelope 102 may include a polymer or binder that is not included in the core 104.

However, according to some embodiments, the division between the envelope 102 and the core 104 may be substantially indistinct. For example, the envelope 102 and the core 104 may be formed of substantially the same materials, with the distinction therebetween being in terms of gelation, cross-linking, and/or hardening. In particular, the envelope 102 and the core 104 may both contain an active agent and a gellant or a gel activator. However, gellant in the envelope 102 may be substantially or completely gelated, while the core 104 may be substantially or completely non-gelated. In addition, the amount of gelation in the envelope 102 may decrease, from the outer surface of the envelope 102 toward and inner surface of the envelope 102.

In other words, the envelope 102 and the core may be 104 distinguished by the relative amounts of gelation thereof. For example, the envelope 102 may be from about 75 to about 100% gelated, such as about 80 to about 95% gelated, or about 85 to 90% gelated. The core 104 may be from about 0 to about 10% gelated, such as about 1 to about 5% gelated, or about 2 to 4% gelated. Herein, a “gelation percentage” may refer to a ratio activated gellant to non-activated gellant.

In various embodiments, the envelope 102 and the core may be 104 distinguished by the viscosities. In particular, the above gelation imparts a higher viscosity to the envelope 102 as compared to that of the core 104. For example, the viscosity of the envelope 102 may be from about 50% to about 1000% higher than the viscosity of the core 104. In particular, the viscosity of the envelope 102 may be from about 65 to about 100% higher than the viscosity of the core 104.

In some embodiments, the tablet 100 may be a compacted solid, and thus, may be non-viscous. In such embodiments, the envelope 104 may be referred to as a shell 104. For example, the tablet 100 may be formed by compressing a tablet mixture in a mold, at a pressure ranging from about 100 psi to about 175 psi. It is believed that higher levels of compression may enable a reduction in the amount of wetting agent and may improve dissolvability.

The shell 104 may be formed by hydrating the outer surface of the tablet 100. For example, the tablet 100 may be, misted, sprayed, or dipped in water or an aqueous solution to form the shell 104. In the alternative, the tablet 100 may be disposed in a humidified environment for a sufficient amount of time to hydrate the outer surface thereof.

Water applied to the outer surface of the tablet 100 may interact with swellable, cross-linkable, hygroscopic polymers, such as guar gum, cationic guar gum, phosphorous modified starch, xanthan gum, and to some extent colloidal oat oligosacharrides. Water may also provide transient cross-linking hydration affects with respect to starch and powdered sucrose. As a result, the shell 104 may be harder than the core 102, and may prevent and/or reduce unwanted breakage of the tablet 100. For example, the shell 104 may have a higher degree of cross-linking than the core 102. The tablet 100 may be dried for from about 12 to about 48 hours, after the water is applied.

In some embodiments, the water applied to the tablet 100 may in the form of an aqueous solution including a hardening agent. For example, the aqueous solution may include witch hazel and/or hydrated urea, which may operate to further solidify the shell 104.

Referring to FIG. 1B, the tablet 110 is similar to the tablet 100 in that it includes an envelope/shell 112 and a core 114. However, the tablet 110 is generally ovoid rather than spherical. As such, the tablet 110 may have higher surface to volume ratio than the tablet 100. In other embodiments, tablets may have irregular spheroid or ovoid shapes, in order to further increase the surface to volume ratios thereof.

Referring to FIG. 1C, the tablet 120 is generally spherical. However, in contrast to the tablets 100, 110, the tablet 120 has a substantially consistent composition/structure. In other words, the tablet may be substantially completely gelated, or the tablet may have an activated binder distributed throughout.

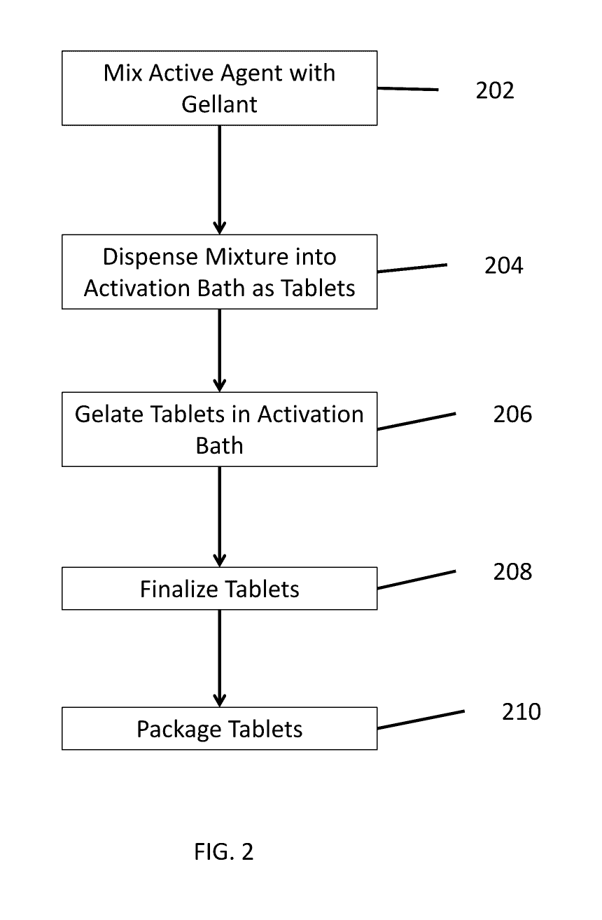

FIG. 2 is a block diagram illustrating a method of forming hygiene product tablets according to various embodiments. Referring to FIG. 2, in step 202, an active agent is mixed with a gellant. The active agent may be a commercially available shampoo, shampoo concentrate, compressed powdered surfactant, or conditioner. In some embodiments, the active agent may be a shaving cream gel or compressed concentrate. The gellant may be an ion-activated polysaccharide as described above. The active agent and gellant may be mixed to form a mixture. The mixture may include from about 0.02 wt % to about 66 wt % gellant, such as about 2.0 wt % to about 5 wt % gellant, based on the total weight of the mixture.

In some embodiments, step 202 may also include adding one or more secondary ingredients to the active agent. In particular, the secondary ingredients may be added to the active agent before or after the active agent is mixed with the gellant.

In step 204, the resultant mixture is shaped into tablets. For example, the mixture may be dispensed as droplets that are dropped into an activation bath. In other embodiments, the mixture may be dispensed from an extruder or the like in tablet form and then dropped into the activation bath. In other embodiments, the mixture may be molded/compressed into tablets and then disposed in the activation bath.

In step 206, the tablets may be reacted with components of the activation bath, such that the tablets become at least partially gelated. The activation bath may be an aqueous solution including gel activators. For example, the water bath may include a solution of calcium chloride or the like. The activation bath may include a gel activator concentration ranging from 0.5 wt % up to a saturation point of the gel activator in the activation bath. For example, the activation bath may include a gel activator concentration of about 1 wt % to about 5 wt %. However, any suitable concentration may be used, so long as the activation bath contains an amount of gel activator sufficient to at least partially gelate the tablets.

In some embodiments a tablet may be in the form of a compacted power. In this case, the activation bath may operate to hydrate the outer surface of the tablet to form an envelope thereon. The hydration may occur with or without the presence of ions, such as calcium ions, in the activation bath.

The tablets may remain in the activation bath for an amount of time sufficient to at least partially gelate the tablets. In particular, each tablet may be disposed in the activation bath for a time period sufficient to form a gelated outer envelope around the tablets, while cores of the tablets remain substantially non-gelated. For example, the tablet may remain in the activation bath for a time period sufficient for the gel activator to penetrate the surface of the tablet and react with the gellant. The time period may be insufficient for the gel activator to completely penetrate the tablet. As such, a gelated envelope structure may be formed on the surface of the tablet, while the core of the tablet remains substantially or completely non-gelated, as shown in tablets 100, 110 of FIGS. 1 and 2. In other words, the gel activator may be localized in the envelope.

In various embodiments, the tablets may be disposed in the activation bath for a time period ranging from about 1 to about 30 seconds, such as a time period ranging from about 2 to about 20 seconds, or about 3 to about 5 seconds. The gelation time of the tablets may be dependent upon the concentration of the gel activator and/or a desired gelation amount. For example, increased gel activator concentration may result in faster/more complete gelation.

In step 208, the tablets may be removed from the activation bath. In particular, any remaining activation bath may be removed from the tablets. For example, the tablets may optionally be rinsed with water or blow dry with air.

In step 210, the tablets may be packaged. For example, the tablets may be packaged in biodegradable or recyclable packs, for example, in polyvinyl alcohol film sheets, polylactic acid bags, starch, or in paper, for example the types of paper used for packaging soap, sugar, or flour. Accordingly, the tablets may be packages in a single dose format with minimal environmental impact.

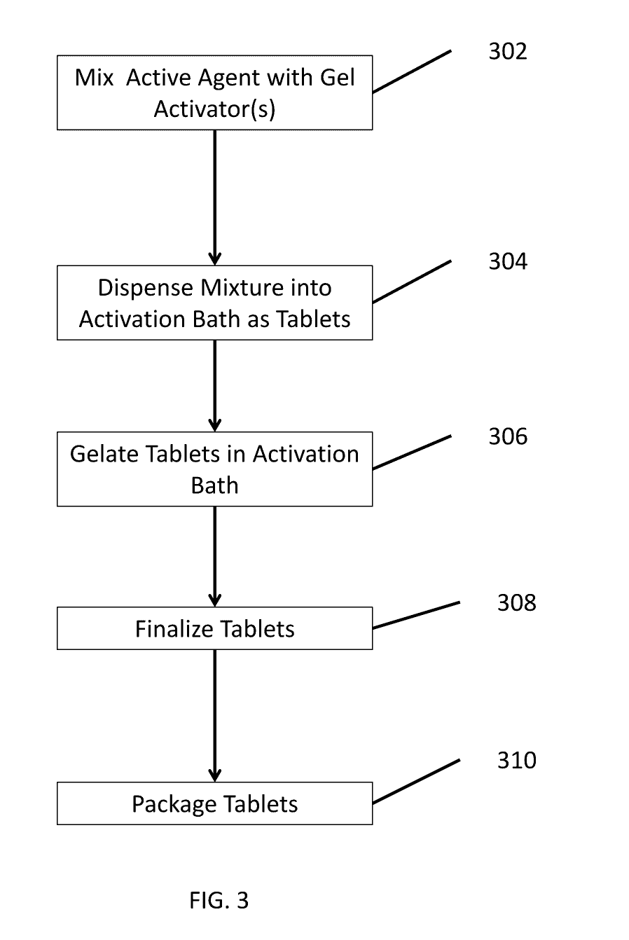

FIG. 3 is a block diagram illustrating a method of forming hygiene product tablets, according to various embodiments. Referring to FIG. 3, in step 302, at least one gel activator is mixed with an active agent. The active agent may be a commercially available shampoo, shampoo concentrate, compressed powdered surfactant, or conditioner. In some embodiments, the active agent may be a shaving cream gel or compressed concentrate. The gel activator may be a calcium salt activator as described above. However, other gel activators may be used according to gellant type.

In some embodiments, step 302 may also include adding one or more secondary ingredients to the active agent. In particular, the secondary ingredients may be added to the active agent before or after the active agent is mixed with the binder.

In step 304, the resultant mixture is dispensed into an activation bath in the form of tablets. For example, the mixture may be dropped into the activation bath in the form of tablets. In other embodiments, the mixture may be molded/compressed into tablets and then disposed in the activation bath. The activation bath may be an aqueous mixture comprising a gellant.

In step 306, each tablet is reacted with the gellant in the activation bath. The gellant may be an ion-activated polysaccharide as described above. The ion-activated polysaccharide may be included in the activation bath at a concentration ranging from about 0.1 to about 10 wt %, such as a concentration ranging from about 2.0 to about 5.0 wt %. However, any suitable concentration may be used.

According to some embodiments, each tablet may remain in the activation bath for a time period sufficient to at least partially gelate the tablet. For example, the tablet may remain in the activation bath for a time period sufficient for the gellant to penetrate into the surface of the tablet and react with the gel activators. The time period may be insufficient for the gellant to completely penetrate the tablet. As such, a gelated envelope or membrane structure may be formed on the surface of the tablet, while the core of the tablet remains substantially or completely non-gelated, as shown in tablets 100, 110 of FIGS. 1 and 2. In other words, the gellant may be localized in the envelope, thereby forming a localized gel.

In step 310, the tablets may be removed from the activation bath. The tablets may be optionally rinsed with water to remove any activation bath remaining thereon.

In step 312, the tablets may be packaged. For example, the tablets may be packaged in biodegradable or recyclable packs, for example, in polyvinyl alcohol film sheets, polylactic acid bags, starch, or in paper, for example the types of paper used for packaging soap, sugar, or flour. Accordingly, the tablets may be packages in a single dose format with minimal environmental impact.

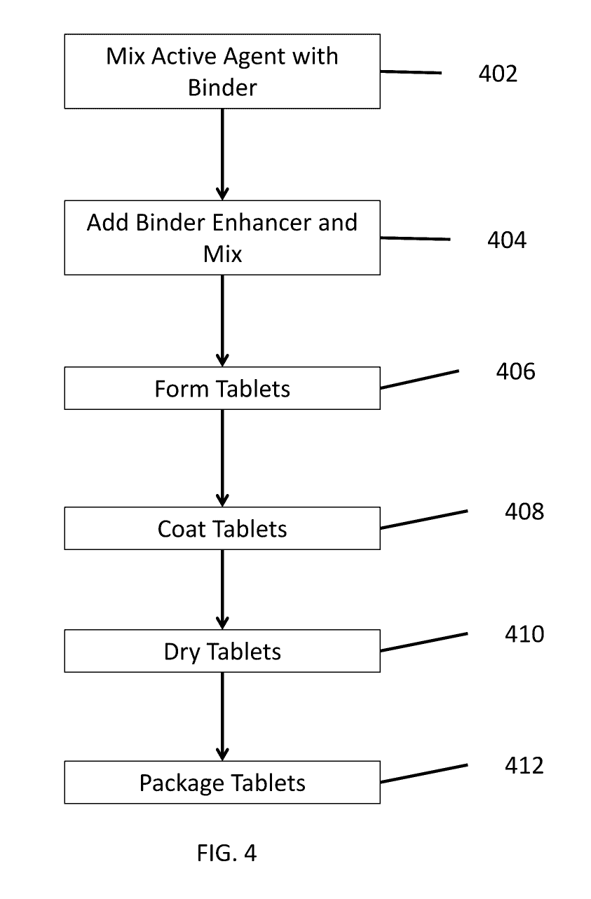

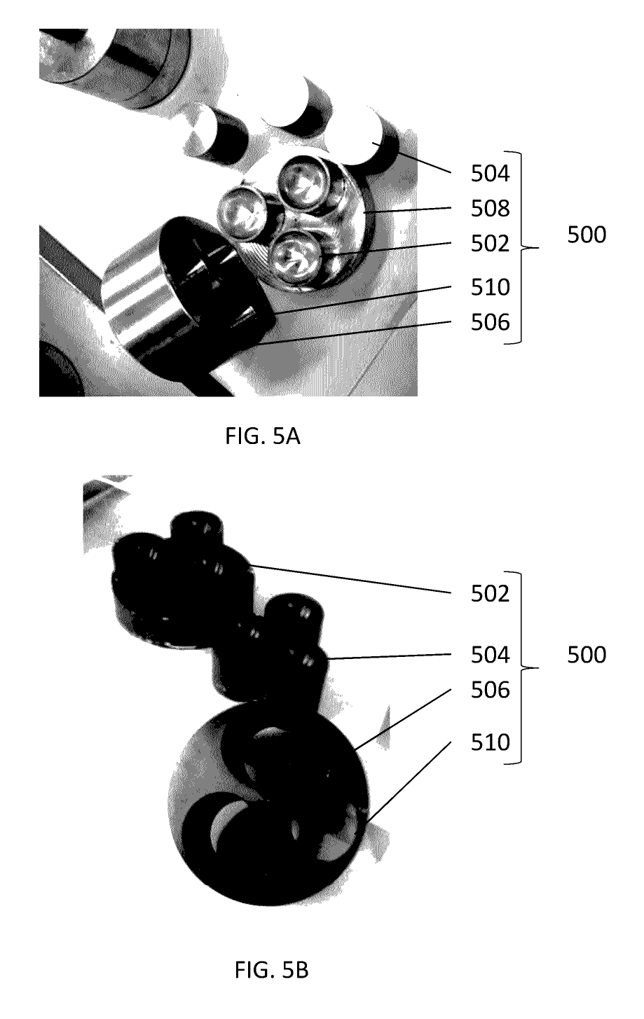

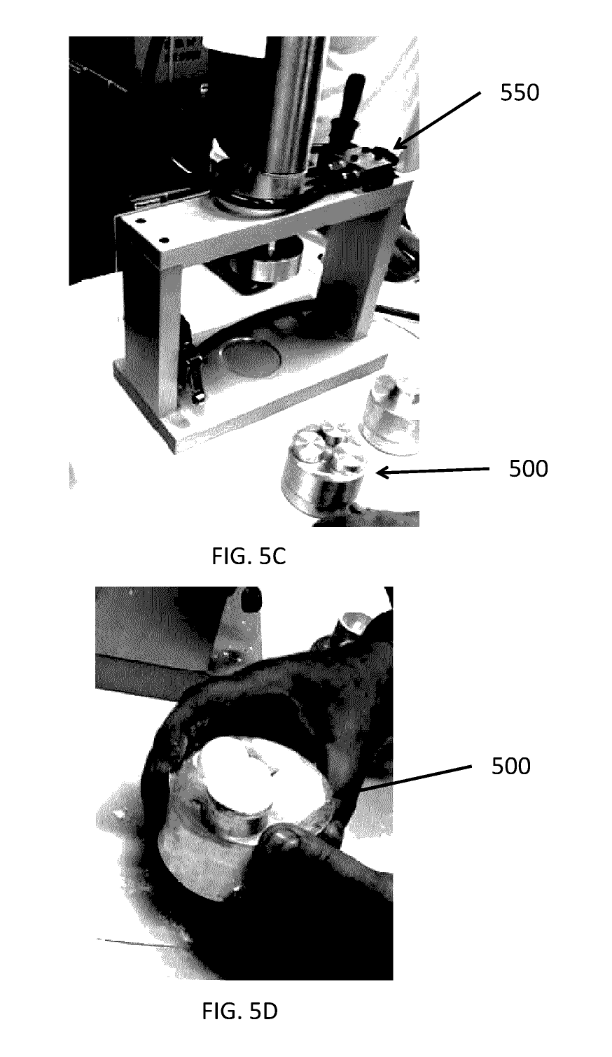

FIG. 4 is a block diagram illustrating a method of forming solid hygiene product tablets, according to various embodiments. FIGS. 5A-5D are photographs illustrating components of a press used in the method of FIG. 4.

Referring to FIG. 4, in step 402 dry tablet components may be mixed in, for example, a high-shear blender. The dry components may include one or more active agents and binders as described above. The binder and active agent may be included at a weight ratio ranging from 70:30 to about 80:20. However, any suitable weight ratio that produces a substantially solid tablet pellet may be used.

The dry components may also include one or more binder enhancers and secondary ingredients, as described above. For example, the binder may include sucrose, the active agent may include one or more non-sulfate surfactants, such as powdered sodium cocoyl isethionate and/or sodium lauryl sulfoacetate, and the secondary ingredients may include hydroxypropyl starch phosphate, guar gum, and/or colorant. The sucrose may be in the form of confectioner's sugar, which may include about 3 wt % corn starch as an anti-caking agent.

In step 404, liquid components may be added to the dry mixture followed by additional blending. The liquid components may be mixed with one another and then slowly added to the dry component mixture, under blending, to form a tablet mixture. The liquid components may include a wetting agent, such as glycerin, and optionally fragrance oil.

In step 406, the tablet mixture may be pressed to form into tablets such as tablet 120 of FIG. 1C. In particular, the tablet mixture may be loaded into a mold, and mold may be compressed to form one or more tablets.

FIGS. 5A and 5B are photographs of a tablet mold 500 useable with various embodiments. FIG. 5C is a photograph of a press 550 used to compress the tablet mold 500, and FIG. 5D is a photograph of tablets after being pressed.

Referring to FIGS. 4, 5A, and 5B, the mold 500 may include a lower mold half 502, and upper mold half 504, and a compression housing 506. The housing 506 may include a cylinder 510 configured to receive the mold halves 502, 504. The mold 500 may include multiple lower and upper mold halves 502, 504, and the housing 506 may include multiple corresponding cylinders 510. In some embodiments, the mold 500 may include a plate 508 to which mold halves, such as the lower mold halves 502, may be attached.

In some embodiments, the mold 500 may be formed of an uncoated metallic material, as shown in FIG. 5A. In this embodiment, a release agent, such as corn starch or the like, may be coated onto the mold halves 502, 504, prior to loading the tablet mixture.

In other embodiments, an anti-stick coating, such as polytetrafluoroethylene, may be formed on the mold 500, as shown in FIG. 5B. In this embodiment, a release agent may be omitted.

In step 406, the tablet mixture may be loaded into the lower mold halves 502, which may then be inserted into the cylinders 510 through the bottom of the housing 506. The upper mold halves 504 may then be inserted into the cylinders 510 through the top of the housing 506, thereby assembling the mold 500, as shown in FIG. 5C.

The mold 500 may be inserted into the press 550. The press 550 may apply a pressure to the mold 500 ranging from about 90 psi to about 200 psi, such as from about 100 to about 180 psi, or from about 105 psi to about 140 psi, to compress the tablet mixture into tablets. The tablets may be removed from the mold 500, as shown in FIG. 5D.

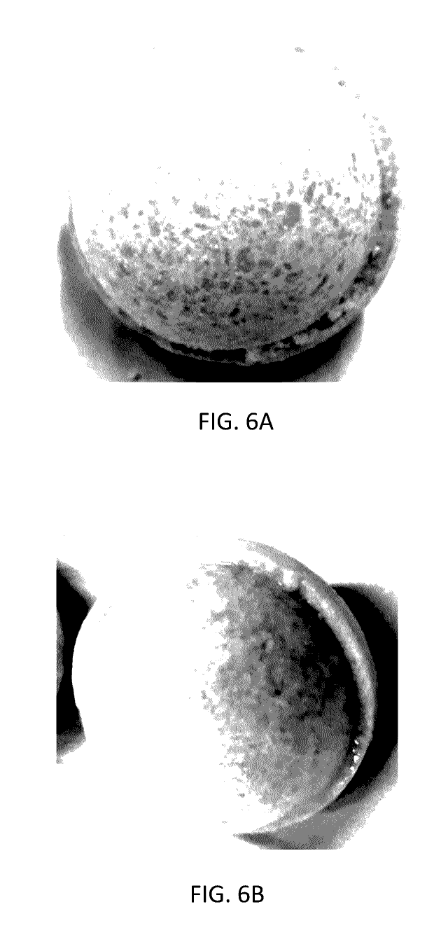

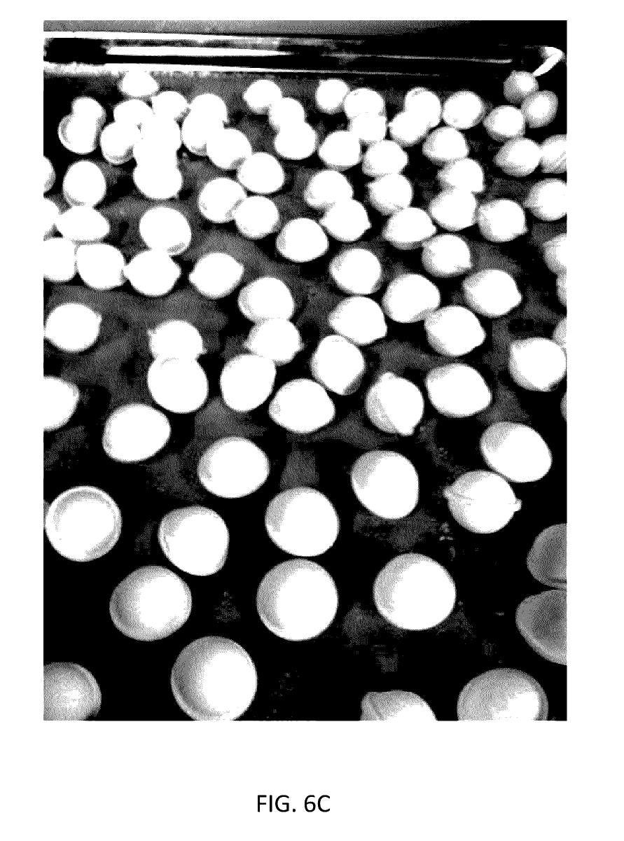

In step 408, the tablets may optionally be coated with water or an aqueous solution including a hardener. For example, the tablets may be misted with water or an aqueous hardener solution, such that a shell may be formed on the tablets. However, in other embodiments, the tablets may be coated by dipping in water or an aqueous hardener solution, or may be subjected to a humidified environment for an amount of time sufficient for water to infiltrate the tablets and form a shell. FIG. 6A is a photograph of a coated tablet, and FIG. 6B is a photograph of a coated tablet.

In step 410, the tablets may be optionally allowed to dry in a room temperature environment, for a time period ranging from about 30 minutes to about 48 hours. In other embodiments, the tablets may be dried in an oven. The drying of the tablets may reduce the tackiness thereof, and allow for easier handling thereof. FIG. 6C is a photograph of tablets being dried.

In step 412, the tablets may be packaged. For example, the tablets may be packaged in biodegradable or recyclable packs, such as in polyvinyl alcohol film sheets, polylactic acid bags, starch, or in paper, for example the types of paper used for packaging soap, sugar, or flour. The packaging may be configured to prevent additional drying or wetting of the tablets. Accordingly, the tablets may be packages in a single dose format with minimal environmental impact.

### EXEMPLARY FORMULATIONS

### Example 1

| Component | Weight Percentage |
|---|---|
| Sucrose | 57.8 |
| Sodium Cocoyl Isethionate | 15.5 |
| Glycerin | 12 |
| Colloidal Oatmeal Powder | 10 |
| Corn Starch | 3 |
| Fragrance Oil | 1 |
| Hydroxypropyl Starch Phosphate | 0.5 |
| Guar Gum | 0.1 |
| Colorant | 0.1 |

### Example 2

| Component | Weight Percentage |
|---|---|
| Sucrose | 68.5 |
| Sodium Cocoyl Isethionate | 15.5 |
| Glycerin | 12.4 |
| Corn Starch | 3 |
| Fragrance Oil | 1 |
| Hydroxypropyl Starch Phosphate | 0.5 |
| Guar Gum | 0.1 |

### Example 3

| Component | Weight Percentage |
|---|---|
| Sucrose | 56.5 |
| Sodium Cocoyl Isethionate | 10 |
| Colloidal Oatmeal Powder | 20 |
| Glycerin | 12 |
| Fragrance Oil | 1 |
| Hydroxypropyl Starch Phosphate | 0.5 |
| Guar Gum | 0.1 |

### Example 4

| Component | Weight Percentage |
|---|---|
| Sucrose | 76 |
| Sodium Cocoyl Isethionate | 10 |
| Glycerin | 12 |
| Fragrance Oil | 1 |
| Hydroxypropyl Starch Phosphate | 0.5 |
| Guar Gum | 0.1 |

The compositions of Examples 1-4 were prepared by separately mixing powdered and liquid ingredients, and then slowing mixing the liquid mixture into the powder mixture. The resultant tablet mixtures were then pressed at from 120 to 130 psi, to form tablets. Some of the tablets were sprayed with an aqueous witch hazel solution.

Tablets of Examples 1-4 were then subjected to a drop test, by dropping the tablets from a height of two feet onto a hard flat surface. The tablets survived the drop test without breakage. Tablets of Examples 1-4 were also subjected to a wash test in which water was used to dissolve each tablet while the tablet was held in hand. The tested tablets easily dissolved, produced a rich lather, and had a smooth texture.

Similar to the examples above, similar combinations of components may be used to form tablets of shaving cream concentrate. For example, a shaving cream gel or concentrate may be mixed with a binder and an optionally a binder enhancer, to produce a shaving cream tablet.

### Comparative Formulations

### Comparative Example 1

| Component | Weight Percentage |
|---|---|
| Sucrose | 76 |
| Sodium Cocoyl Isethionate | 19 |
| Propylene Glycol | 5 |

### Comparative Example 2

| Component | Weight Percentage |
|---|---|
| Sucrose | 76 |
| Sodium Cocoyl Isethionate | 19 |
| Glycerin | 5 |

### Comparative Example 3

| Component | Weight Percentage |
|---|---|
| Sucrose | 76 |
| Sodium Cocoyl Isethionate | 19 |
| Aqueous Witch Hazel | 5 |

### Comparative Example 4

| Component | Weight Percentage |
|---|---|
| Sucrose | 74 |
| Sodium Cocoyl Isethionate | 18.5 |
| Hydroxypropyl Starch Phosphate | 1 |
| Guar Gum | 0.5 |
| Glycerin | 5 |
| Fragrance | 1 |

### Comparative Example 5

| Component | Weight Percentage |
|---|---|
| Sucrose | 64 |
| Sodium Cocoyl Isethionate | 16 |
| Sodium Lauryl Sulfoacetate | 10 |
| Coco Powder | 10 |
| Hydroxypropyl Starch Phosphate | 0.5 |
| Guar Gum | 0.5 |
| Glycerin | 7 |
| Fragrance | 1 |

### Comparative Example 6

| Component | Weight Percentage |
|---|---|
| Sucrose | 56 |
| Sodium Cocoyl Isethionate | 14 |
| Sodium Lauryl Sulfoacetate | 10 |
| Hydroxypropyl Starch Phosphate | 0.5 |
| Guar Gum | 0.1 |
| Glycerin | 7 |
| Fragrance | QS. |

### Comparative Example 7

| Component | Weight Percentage |
|---|---|
| Sucrose | 56 |
| Sodium Cocoyl Isethionate | 14 |
| Sodium Lauryl Sulfoacetate | 10 |
| Arrow Root | 10 |
| Hydroxypropyl Starch Phosphate | 0.5 |
| Guar Gum | 0.1 |
| Glycerin | 7 |
| Fragrance | QS. |

### Comparative Example 8

| Component | Weight Percentage |
|---|---|
| Sucrose | 75 |
| Sodium Cocoyl Isethionate | 10 |
| Sodium Lauryl Sulfoacetate | 10 |
| Hydroxypropyl Starch Phosphate | 0.5 |
| Glycerin | 7 |
| Fragrance | QS. |

### Comparative Example 9

| Component | Weight Percentage |
|---|---|
| Sucrose | 75 |
| Sodium Cocoyl Isethionate | 10 |
| Sodium Lauryl Sulfoacetate | 10 |
| Hydroxypropyl Starch Phosphate | 0.5 |
| Glycerin | 10 |
| Fragrance | QS. |

### Comparative Example 10

| Component | Weight Percentage |
|---|---|
| Sucrose | 70 |
| Sodium Cocoyl Isethionate | 10 |
| Sodium Lauryl Sulfoacetate | 10 |
| Hydroxypropyl Starch Phosphate | 0.5 |
| Glycerin | 10 |
| Fragrance | QS. |

Tablets were formed using the compositions of Comparative Examples 1-10 using methods similar to those used to form Examples 1-4. However, the tablets suffered from various deficiencies. For example, tablets of Comparative Examples 1-9 failed to form a rigid tablet structure (e.g., had a gooey texture) and/or failed to produce a sufficient amount of lather. The tablets of Comparative Examples 10 was firm enough to form a tablet, but failed the drop test.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the scope of the invention. Thus, the present invention is not intended to be limited to the aspects and/or embodiments shown herein but is to be accorded the widest scope consistent with the following claims and the principles and novel features disclosed herein.

What is claimed is:

1. A single-use hygiene product tablet comprising, based on the total weigh of the tablet:
a binder in an amount ranging from about 12 wt % to about 20 wt %;
an active agent in an amount ranging from about 55 wt % to about 70 wt %; and
a non-aqueous wetting agent in an amount ranging from about 7 wt % to about 18 wt %.

2. The tablet of claim 1, further comprising a binder enhancer comprising a cross-linkable, hygroscopic polymer in an amount ranging from about 0.05 wt % to about 0.3 wt %.

3. The tablet of claim 2, wherein the hygroscopic polymer comprises guar gum, cationic guar gum, hydroxyproply starch phosphate, xanthan gum, or any combination thereof.

4. The tablet of claim 2, wherein the binder enhancer further comprises a modified starch in an amount ranging from about 0.3 wt % to about 1 wt %.

5. The tablet of claim 4, wherein the modified starch comprises hydroxypropyl starch phosphate, sodium starch glycolate, or any combination thereof.

6. The tablet of claim 1, wherein the active agent comprises sodium cocoyl isethionate, sodium lauryl sulfoacetate, alpha olefin sulfonate, or any combination thereof.

7. The tablet of claim 1, wherein the wetting agent comprises glycerin, propylene glycol, dipropylene glycol, N-acetyl diglycoamine, safflower oil, or any combination thereof.

8. The tablet of claim 1, wherein the tablet further comprises coconut milk powder, arrow root powder, colloidal oatmeal powder, or any combination thereof, in an amount ranging from about 5 wt % to about 18 wt %.

9. The tablet of claim 1, wherein the tablet comprises:
a core; and
a shell surrounding the core, the shell having a higher degree of cross-linking than the core.

10. The tablet of claim 9, wherein:
the shell comprises witch hazel, hydrated urea, or a combination thereof; and
the core is substantially free of witch hazel.

11. The tablet of claim 1, wherein the tablet is formed by compression in a mold, at a pressure ranging from about 100 psi to about 180 psi.

12. A method of forming a hygiene product tablet, the method comprising:
forming a first mixture by mixing a binder and an active agent;
forming a second mixture by mixing a non-aqueous wetting agent into the first mixture;
compressing the second mixture in a mold at from about 100 psi to about 180 psi, to form a tablet; and
coating the tablet with water or an aqueous mixture comprising a hardening agent.

13. The method of claim 12, wherein the forming a first mixture further comprises mixing a cross-linkable, hygroscopic polymer with the binder and an active agent.

14. The method of claim 12, wherein:
the hygroscopic polymer comprises guar gum, cationic guar gum, hydroxyproply starch phosphate, xanthan gum, or any combination thereof; and
the wetting agent comprises glycerin, propylene glycol, dipropylene glycol, N-acetyl diglycoamine, safflower oil, or any combination thereof.

15. The method of claim 12, wherein the forming a first mixture further comprises mixing hydroxypropyl starch phosphate, sodium starch glycolate, or any combination thereof, with the binder and active agent.

16. The method of claim 12, wherein the forming a first mixture further comprises mixing coconut milk powder, arrow root powder, colloidal oatmeal powder, or any combination thereof, with the binder and active agent.

17. The method of claim 12, wherein the forming a first mixture further comprises mixing hydroxypropyl starch phosphate, sodium starch glycolate, or any combination thereof, with the binder and active agent.

18. The method of claim 12, wherein the active agent comprises sodium cocoyl isethionate, sodium lauryl sulfoacetate, alpha olefin sulfonate, or any combination thereof.

19. The method of claim 12, wherein:
the coating comprises coating the tablet with an aqueous mixture comprising witch hazel, hydrated urea, or any combination thereof; and
the mold is coated with polytetrafluoroethylene.

20. A single-use hygiene product tablet comprising:
a binder;
a sulfate-free surfactant;
a non-aqueous wetting agent; and
a cross-linkable, hygroscopic polymer,
wherein the tablet comprises a core and a shell surrounding the core, the shell having a higher degree of cross-linking than the core, and
wherein the tablet is formed by compression in a mold, at a pressure ranging from about 100 psi to about 180 psi.
